SEQUENCE LISTING (13 sequences)

SEQ ID NO: 1 - protein, 24 aa, Artificial Sequence
  Synthetic polypeptide
QGQVQHLQAAFSQYKKVELFPKGG

SEQ ID NO: 2 - protein, 87 aa, Artificial Sequence
  Synthetic polypeptide
MAKTHHHHHHQGQVQHLQAAFSQYKKVELFPKGGQGQVQHLQAAFSQYKKVELFPKGGQGQVQHLQAAFSQYKKVELFPKGGYYYYC

SEQ ID NO: 3 - protein, 4 aa, Artificial Sequence
  Synthetic polypeptide
YYYY

SEQ ID NO: 4 - protein, 11 aa, Artificial Sequence
  Synthetic polypeptide
AKTKKVELFPK

SEQ ID NO: 5 - protein, 12 aa, Artificial Sequence
  Synthetic polypeptide
AKTKKVELFPKC

SEQ ID NO: 6 - protein, 60 aa, Artificial Sequence
  Synthetic polypeptide
  misc_feature (3)..(60): may be absent
GSGSGSGSGSGSGSGSGSGSGSGSGSGSGSGSGSGSGSGSGSGSGSGSGSGSGSGSGSGS

SEQ ID NO: 7 - protein, 90 aa, Artificial Sequence
  Synthetic polypeptide
  misc_feature (4)..(90): may be absent
GGSGGSGGSGGSGGSGGSGGSGGSGGSGGSGGSGGSGGSGGSGGSGGSGGSGGSGGSGGSGGSGGSGGSGGSGGSGGSGGSGGSGGSGGS

SEQ ID NO: 8 - protein, 120 aa, Artificial Sequence
  Synthetic polypeptide
  misc_feature (5)..(120): may be absent
GGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGS

SEQ ID NO: 9 - protein, 150 aa, Artificial Sequence
  Synthetic polypeptide
  misc_feature (6)..(150): may be absent
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 10 - protein, 30 aa, Artificial Sequence
  Synthetic polypeptide
  misc_feature (2)..(30): may be absent
GGGGGGGGGGGGGGGGGGGGGGGGGGGGGG

SEQ ID NO: 11 - protein, 150 aa, Artificial Sequence
  Synthetic polypeptide
  misc_feature (6)..(150): may be absent
EAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAK

SEQ ID NO: 12 - protein, 120 aa, Artificial Sequence
  Synthetic polypeptide
  misc_feature (5)..(120): may be absent
SGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGS

SEQ ID NO: 13 - protein, 60 aa, Artificial Sequence
  Synthetic polypeptide
  misc_feature (1)..(1): Xaa can be any naturally occurring amino acid
  misc_feature (3)..(3): Xaa can be any naturally occurring amino acid
  misc_feature (5)..(5): Xaa can be any naturally occurring amino acid
  misc_feature (7)..(7): Xaa can be any naturally occurring amino acid
  misc_feature (9)..(9): Xaa can be any naturally occurring amino acid
  misc_feature (11)..(11): Xaa can be any naturally occurring amino acid
  misc_feature (13)..(13): Xaa can be any naturally occurring amino acid
  misc_feature (15)..(15): Xaa can be any naturally occurring amino acid
  misc_feature (17)..(17): Xaa can be any naturally occurring amino acid
  misc_feature (19)..(19): Xaa can be any naturally occurring amino acid
  misc_feature (21)..(21): Xaa can be any naturally occurring amino acid
  misc_feature (23)..(23): Xaa can be any naturally occurring amino acid
  misc_feature (25)..(25): Xaa can be any naturally occurring amino acid
  misc_feature (27)..(27): Xaa can be any naturally occurring amino acid
  misc_feature (29)..(29): Xaa can be any naturally occurring amino acid
  misc_feature (31)..(31): Xaa can be any naturally occurring amino acid
  misc_feature (33)..(33): Xaa can be any naturally occurring amino acid
  misc_feature (35)..(35): Xaa can be any naturally occurring amino acid
  misc_feature (37)..(37): Xaa can be any naturally occurring amino acid
  misc_feature (39)..(39): Xaa can be any naturally occurring amino acid
  misc_feature (41)..(41): Xaa can be any naturally occurring amino acid
  misc_feature (43)..(43): Xaa can be any naturally occurring amino acid
  misc_feature (45)..(45): Xaa can be any naturally occurring amino acid
  misc_feature (47)..(47): Xaa can be any naturally occurring amino acid
  misc_feature (49)..(49): Xaa can be any naturally occurring amino acid
  misc_feature (51)..(51): Xaa can be any naturally occurring amino acid
  misc_feature (53)..(53): Xaa can be any naturally occurring amino acid
  misc_feature (55)..(55): Xaa can be any naturally occurring amino acid
  misc_feature (57)..(57): Xaa can be any naturally occurring amino acid
  misc_feature (59)..(59): Xaa can be any naturally occurring amino acid
  misc_feature (13)..(60): may be absent
XPXPXPXPXPXPXPXPXPXPXPXPXPXPXPXPXPXPXPXPXPXPXPXPXPXPXPXPXPXP

(19) United States
(12) Patent Application Publication
(10) Pub. No.: US 2019/0314260 A1
(43) Pub. Date: Oct. 17, 2019

(54) ENGINEERED TREATMENTS FOR HAIR REPAIR AND LONG-LASTING COLOR RETENTION

(71) Applicant: Massachusetts Institute of Technology, Cambridge, MA (US)

(72) Inventors: Bradley David Olsen, Arlington, MA (US); Sieun Kim, Waltham, MA (US)

(73) Assignee: Massachusetts Institute of Technology, Cambridge, MA (US)

(21) Appl. No.: 16/382,632

(22) Filed: Apr. 12, 2019

Related U.S. Application Data
(60) Provisional application No. 62/657,344, filed on Apr. 13, 2018.

(51) Int. Cl. A61K 8/65; C07K 14/47; A61Q 5/00; A61Q 5/10; C12N 15/70

(52) U.S. Cl. CPC A61K 8/65; C07K 14/4741; C12N 15/70; A61Q 5/10; A61Q 5/002

(57) ABSTRACT

Disclosed herein are engineered constructs that have keratin-binding functionality, and uses thereof for hair treatment.

### RELATED APPLICATIONS

This application claims the benefit under 35 U.S.C. § 119(e) of U.S. provisional application No. 62/657,344, filed Apr. 13, 2018, which is incorporated by reference herein in its entirety.

### FIELD

Disclosed herein are engineered constructs that have keratin-binding functionality, and uses thereof for hair treatment.

### BACKGROUND

Hair sustains damage from environmental factors, ageing, washing, coloring and styling. Repeated washing results in lifted cuticles, while heat damage from drying, straightening, or curling hair leads to dehydrated, frizzy hair and split ends. Ageing and UV exposure culminate in a loss of melanin, leaving hair bleached or grey. Hair colorant and relaxant products raise cuticles using harsh chemicals leading to hair loss and/or breakage, and cause skin damage and irritation. Accordingly, there is a need for hair treatments and products that can protect and/or repair damaged hair. There is also a need for products that color hair without the use of harsh chemicals that cause hair damage and harm skin.

### SUMMARY

Disclosed herein are engineered constructs that have keratin-binding functionality, and uses thereof for hair treatment. For example, engineered keratin-binding constructs comprising at least one keratin-binding molecule are disclosed. In some embodiments, the engineered keratin-binding construct comprises one or more of each of two or more types of keratin-binding molecule. In some embodiments, one or more of the keratin-binding molecules are keratin-binding proteins. For example, the keratin-binding protein can comprise 1 to 5 repeats of the amino acid sequence QGQVQHLQAAFSQYKKVELFPKGG (SEQ ID NO: 1). In some embodiments, the engineered keratin-binding construct comprises the formula Xn-Ym, where X is one type of keratin-binding molecule, and Y is a different type of keratin-binding molecule, n=0−20 and m=0−20, and n and m cannot both be 0.

In some aspects, the keratin-binding constructs disclosed herein comprise a site for bioconjugation. In some embodiments, the site for bioconjugation is for bioconjugation of a lipid. In some embodiments, the site for bioconjugation is located at or near an end of the engineered keratin-binding construct. In one example, at least one keratin-binding molecule is a protein, and the site for bioconjugation is located at the N-terminus of the protein or within 5 amino acid residues from the N-terminus of the protein. In some embodiments, the site for bioconjugation comprises a pyridoxal 5-phosphate (PLP) reactive site. For example, the PLP reactive site is the amino acid sequence alanine-lysine-threonine (AKT).

In some examples of the engineered keratin-binding construct disclosed herein, the construct further comprises a keratin conjugation site. In some embodiments, the keratin conjugation site comprises one or more cysteines. In some embodiments, the keratin conjugation site may be located at or near an end of the engineered keratin-binding construct. For example, in some aspects, at least one keratin-binding molecule is a protein, and the keratin conjugation site is located at the C-terminus of the protein. In some embodiments, at least one keratin-binding molecule is a protein, and one or more cysteines is part of a single polypeptide chain with the keratin-binding molecule.

In some aspects the disclosure relates to engineered keratin-binding constructs that further contains a dye that imparts color to the molecule. In some embodiments, this dye comprises a melanin precursor. In some embodiments, the melanin precursor can comprise one or more tyrosines. By way of example, in some embodiments, the melanin precursor includes between 4-20 tyrosines. In some embodiments of the keratin-binding constructs disclosed herein, at least one keratin-binding molecule is a protein, and one or more tyrosines is part of a single polypeptide chain with the keratin-binding molecule. In some aspects, the keratin-binding construct further comprises melanin or a hair dye.

In some aspects disclosed herein, the keratin-binding constructs comprise a lipid. In some embodiments, the lipid is conjugated to the site for bioconjugation. For example, the site for bioconjugation can be the amino acid sequence AKT, and the lipid is conjugated to the lysine of the amino acid sequence AKT. Any suitable lipids can be conjugated to the keratin-binding molecules disclosed herein. Examples include, but are not limited to, myristoleic acid/alcohol/amine, palmitoleic acid/alcohol/amine, sapienic acid/alcohol/amine, oleic acid/alcohol/amine, elaidic acid/alcohol/amine, vaccenic acid/alcohol/amine, linoleic acid/alcohol/amine, linoelaidic acid/alcohol/amine, alpha linolenic acid/alcohol/amine, arachidonic acid/alcohol/amine, eicosapentaenoic acid/alcohol/amine, erucic acid/alcohol/amine, caprylic acid/alcohol/amine (octanoic acid/alcohol/amine), lauric acid/alcohol/amine, myristic acid/alcohol/amine, palmitic acid/alcohol/amine, lignoceric acid/alcohol/amine, arachidic acid/alcohol/amine, stearic acid/alcohol/amine, and sphingolipids including ceramide, sphingosine, sphingomyelin, alpha cerebroside, gangliosides, sulfatides, and phytosphingosine.

Also disclosed are compositions comprising the engineered keratin-binding construct. These compositions may comprise one or more cosmetic ingredients. Any suitable cosmetic ingredients can be included in the composition. Suitable cosmetic ingredients include, but are not limited to, surfactants, preservatives, emulsifiers, softeners, moisturizers, humectants, hydrolyzed proteins, reconstructors, acidifiers, acidity regulators, detanglers, polymers, glossers, lubricants, sequestrants, antistatic agents, sunscreens, thermal protectors, conditioners, buffers, stabilizers, thickeners, salts, emollients, antioxidants, alcohols, polysorbates, PEGs, polyquaternium polymers, quarternary ammonium compounds, fragrances, dyes or colors, oils, esters, fatty acids, bioactive additives, food products, silicones, and water. In some embodiments, the compositions may include one or more of citric acid, PEG-150, PEG(20), PEG(80), ammonium chloride, ascorbates, straight-chain alkyl benzene sulfonates (e.g., ammonium lauryl sulfate), sodium lauryl sulfate, sodium laureth sulfate, amodimethicone, dimethiconol, dimethicone, cyclomethicone, panthenol, cetyl alcohol, oleyl alcohol, stearyl alcohol, sodium lauroamphoacetate, glycol, quarternium-15, polypuaternium-10, Di-PPG-2 myreth-10 adipate, methylisothiazolinone, stearyldimoniumhydroxypropyl, hydroxypropyltrimonium, AMP-isostearoyl, PG-propyl silanetriol, PVP crosspolymer, ethyldimonium ethosulfate, triticum vulgare (wheat), hordeum vulgare (barley), secale cereale (rye), or avena sativa (oats), including any oil, protein, hydrosylate, or other extract from any part of the plant, hydrolyzed wheat protein or hydrolyzed wheat starch, hydrolyzed vegetable protein, glycerine, propylene glycol, stearalkonium chloride, disteardimonium chloride, quaternium-5 or quaternium-18, polyquaternium-10, cetrimonium chloride, cyclodextrin, tocopherol, tocopheryl acetate, maltodextrin, dextrin, dextrin palmitate, or (hydrolyzed) malt extract, yeast extract, grain extract, prolamine, amino peptide complex, beta glucan, phytosphingosine extract, parabens (e.g., methylparaben, ethylparaben, butylparaben), zinc pyrithione, cocamidopropyl betaine, PEG-5 cocamide, ammonium xylenesulfonate, glycerol stearate, glycol distearate, isopropyl palmitate, erithritol, sodium PCA, hyaluronic acid, sorbitol, fructose, fatty alcohols, ethylhexyl methoxycinnamate, benzophenone, polyamide-2, salicylates, PABA, and dimethylparamidopropyl laurdimonium tosylate.

Also provided herein in some aspects are methods for producing the keratin-binding molecules, nucleic acids encoding any one of the keratin-binding molecules, vectors that can comprise any one or more of the nucleic acids provided herein, and related host cells. In some examples, the host cell is a bacterial cell or a yeast cell. In some embodiments, the host cell is an E. coli cell.

Further disclosed herein are methods of improving or repairing damage to hair. In some embodiments, the method comprises applying the engineered keratin-binding constructs and/or compositions disclosed herein to the hair for a time sufficient to improve or repair the damage to the hair. In some embodiments, the method includes leaving the engineered keratin-binding constructs and/or compositions disclosed herein on the hair without rinsing.

In some embodiments, the method includes leaving the engineered keratin-binding constructs and/or compositions disclosed herein for a set period of time, for example, 1, 2, 3, 4, 5, 6, 7, 8, 9, 10, 10-15, 15-20, 20-25, 25-30, 30-45, 45-60, 60-120, 120-180, or more than 180 minutes. In some embodiments, the method includes rinsing the hair to remove excess of the engineered keratin-binding construct. In some embodiments, the method comprises adding or including the engineered keratin-binding constructs and/or components disclosed herein into daily or frequent use products including but not limited to shampoos, conditioners, gels, mousses, pomades, anti-frizz agents, sprays, or hair dyeing products that may be applied to the hair as part of customary hair care procedures including washing, conditioning, dyeing, drying, and styling. In some embodiments, the method may be performed in a salon. In some embodiments, the method may be suitable for home use.

Also provided herein in some aspects are methods of coloring or dyeing hair comprising applying the engineered keratin-binding constructs or the compositions disclosed herein to the hair for a time sufficient to color or dye the hair. In some examples, the engineered keratin-binding comprises melanin or a hair dye molecule conjugated to the at least one keratin-binding molecule. In some embodiments, the method includes leaving the engineered keratin-binding constructs and/or compositions disclosed herein on the hair without rinsing. In some embodiments, the method includes leaving the engineered keratin-binding constructs and/or compositions disclosed herein for a set period of time, for example, 1, 2, 3, 4, 5, 6, 7, 8, 9, 10, 10-15, 15-20, 20-25, 25-30, 30-45, 45-60, 60-120, 120-180, or more than 180 minutes. In some embodiments, the method includes rinsing the hair to remove excess of the engineered keratin-binding construct. In some embodiments, the method comprises adding or including the engineered keratin-binding constructs and/or components disclosed herein into daily or frequent use products including but not limited to shampoos, conditioners, gels, mousses, pomades, anti-frizz agents, sprays, or hair dyeing products that may be applied to the hair as part of customary hair care procedures including washing, conditioning, dyeing, drying, and styling. In some embodiments, the method may be performed in a salon. In some embodiments, the method may be suitable for home use.

In some aspects of the methods disclosed herein, the engineered keratin-binding construct or the composition is applied to the hair of a subject. For example, in some embodiments, the engineered keratin-binding construct or the composition is applied to the head hair of a subject. In some embodiments, the subject is a human subject.

Further disclosed are methods for producing an engineered keratin-binding construct. In some aspects, engineered keratin-binding construct is expressed in a cell or chemically synthesized. In some embodiments, the engineered keratin-binding construct is expressed in a bacterial cell or a yeast cell. In some embodiments, the engineered keratin-binding construct is expressed in an E. coli cell. In some embodiments, the keratin-binding construct is synthesized using a peptide synthesizer.

Also provided herein are methods for producing an engineered keratin-binding construct comprising a lipid. In some embodiments, the method comprises expressing the engineered keratin-binding construct disclosed herein in a cell or chemically synthesizing the engineered keratin-binding construct disclosed herein, and conjugating a lipid to the engineered keratin-binding construct. In some aspects, the lipid is conjugated to the engineered keratin-binding construct at a site for bioconjugation contained in the engineered keratin-binding construct. In some aspects of the method, the site for bioconjugation is the amino acid sequence AKT, and the lipid is conjugated to the lysine of the amino acid sequence AKT. In some embodiments, the method includes conjugating a lipid to the engineered keratin-binding construct by contacting the engineered keratin-binding construct with pyridoxal 5-phosphate (PLP) to form a ketone or aldehyde; and contacting the ketone or aldehyde with a aminooxy-lipid, optionally in the presence of aniline, to form a keratin-binding construct—lipid conjugate.

Also disclosed herein are methods for preparing hair dye compositions. In some aspects, the method comprises expressing the engineered keratin-binding construct disclosed herein in a cell or chemically synthesizing the engineered keratin-binding constructs disclosed herein. In some embodiments, the engineered keratin-binding construct comprises a melanin precursor, and the engineered keratin-binding construct is contacted with tyrosinase to convert the melanin precursor to melanin. In some examples, the tyrosinase is coexpressed in the cell. Alternately, in some examples, the engineered keratin-binding construct is isolated from the cell prior to contacting the engineered polypeptide construct with the tyrosinase. In some embodiments, the cell is a bacterial or yeast cell. In some embodiments, the cell is an E. coli cell. In some embodiments, the method may include contacting the engineered keratin-binding construct with pyridoxal 5-phosphate (PLP) to form ketone or aldehyde; and contacting the ketone or aldehyde with an aminooxy-lipid, optionally in the presence of aniline, to form a keratin-binding construct—lipid conjugate.

Further disclosed herein are methods for preparing a hair product composition. In some aspects the methods comprises combining one or more engineered keratin-binding constructs disclosed herein with one or more cosmetic ingredients. Suitable cosmetic ingredients include, but are not limited to, surfactants, preservatives, emulsifiers, softeners, moisturizers, humectants, hydrolyzed proteins, reconstructors, acidifiers, acidity regulators, detanglers, polymers, glossers, lubricants, sequestrants, antistatic agents, sunscreens, thermal protectors, conditioners, buffers, stabilizers, thickeners, salts, emollients, antioxidants, alcohols, polysorbates, PEGs, polyquaternium polymers, quarternary ammonium compounds, fragrances, dyes or colors, oils, esters, fatty acids, bioactive additives, food products, silicones, and water. In some embodiments, the compositions may include one or more of citric acid, PEG-150, PEG(20), PEG(80), ammonium chloride, ascorbates, straight-chain alkyl benzene sulfonates (e.g., ammonium lauryl sulfate), sodium lauryl sulfate, sodium laureth sulfate, amodimethicone, dimethiconol, dimethicone, cyclomethicone, panthenol, cetyl alcohol, oleyl alcohol, stearyl alcohol, sodium lauroamphoacetate, glycol, quarternium-15, polypuaternium-10, Di-PPG-2 myreth-10 adipate, methylisothiazolinone, stearyldimoniumhydroxypropyl, hydroxypropyltrimonium, AMP-isostearoyl, PG-propyl silanetriol, PVP crosspolymer, ethyldimonium ethosulfate, triticum vulgare (wheat), hordeum vulgare (barley), secale cereale (rye), or avena sativa (oats), including any oil, protein, hydrosylate, or other extract from any part of the plant, hydrolyzed wheat protein or hydrolyzed wheat starch, hydrolyzed vegetable protein, glycerine, propylene glycol, stearalkonium chloride, disteardimonium chloride, quaternium-5 or quaternium-18, polyquaternium-10, cetrimonium chloride, cyclodextrin, tocopherol, tocopheryl acetate, maltodextrin, dextrin, dextrin palmitate, or (hydrolyzed) malt extract, yeast extract, grain extract, prolamine, amino peptide complex, beta glucan, phytosphingosine extract, parabens (e.g., methylparaben, ethylparaben, butylparaben), zinc pyrithione, cocamidopropyl betaine, PEG-5 cocamide, ammonium xylenesulfonate, glycerol stearate, glycol distearate, isopropyl palmitate, erithritol, sodium PCA, hyaluronic acid, sorbitol, fructose, fatty alcohols, ethylhexyl methoxycinnamate, benzophenone, polyamide-2, salicylates, PABA, and dimethylparamidopropyl laurdimonium tosylate.

These and other aspects of the engineered constructs and methods are further described below.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein. It is to be understood that the data illustrated in the drawings in no way limit the scope of the disclosure.

FIG. 1A shows a 3-D depiction and amino acid sequence of an embodiment of a keratin-binding molecule, including the N-terminal amine site as a selective site for chemistry, tyrosine to melanin formation for color dyeing, and C-terminal cysteine as a conjugation site to keratin protein in hair. FIG. 1A also shows a Dot Blot and SDS-PAGE gel depicting the successful expression of KBPY, and a graph showing the results of an MTT assay, in which NIH3T3 fibroblast cell viability was increased in the presence of KBP and KBPY. Below the line, FIG. 1A also shows a schematic of an E. coli cell co-expressing keratin-binding protein and tyrosinase, and a schematic outlining the conversion of tyrosine in KBP to melanin in the presence of tyrosinase.

FIG. 1B is a schematic diagram showing disulfide bonding and electrostatic interactions between an exemplary engineered keratin-binding construct and keratin proteins in hair. The use of the engineered keratin-binding construct fills holes and glues cuticles to provide repair of damaged hair, resulting in healthy hair.

FIG. 2A shows the formation of functionalized lipids. Specifically shown are the formation of aminooxy-ceramide from C2-ceramide, and aminooxy-oleic alcohol from oleyl alcohol.

FIG. 2B shows an exemplary synthesis scheme for producing an aminooxy lipid.

FIG. 3 shows bioconjugation of lipid to keratin-binding protein via oxime ligation.

FIGS. 4A and 4B show comparisons of healthy and damaged hair. FIG. 4A shows side-by-side digital images of healthy hair (left) and damaged hair (right). FIG. 4B shows side-by-side atomic force microscope (AFM) images of healthy hair (left) and damaged hair (right). Damaged hair shows bleached color and cuticle loss.

FIG. 5 shows a keratin binding domain absorption test performed with two keratin binding domains, “Peptide1” and “Peptide2”, each conjugated to fluorescein isothiocyanate (FITC). The peptide-FITC conjugate is not seen on healthy hair because the hydrophobic surface of healthy hair cuticles prevents binding of the peptide. However, peptide-FITC conjugates are shown to be absorbed in damaged hair because the peptide bonds to exposed keratin proteins on damaged hair shafts. In addition, FIG. 5 shows that both peptides exhibit strong binding affinity to the damaged hair but Peptide 2 shows higher affinity as compared to Peptide 1. This is due to binding of the C-terminal cysteine present on Peptide 2 to the exposed keratin protein on the damaged hair.

FIGS. 6A-6C show keratin-binding protein expression in various vectors and strains. FIG. 6A shows a matrix of tested plasmids, cells and keratin-binding proteins. Expression of the keratin-binding proteins with high yield by different combinations of cell lines and plasmids is shown in a Dot Blot (FIG. 6B) and an SDS-PAGE gel (FIG. 6C).

FIG. 7 shows a schematic of a 2-step bioconjugation scheme, and an SDS-PAGE gel showing keratin-binding protein conjugated with dye.

FIG. 8 shows fluorescence microscopy images of healthy hair and damaged hair. Keratin-binding protein was covalently conjugated with fluorescent dye. The graph shows quantitation of fluorescent intensity of the images.

FIG. 9 shows fluorescence microscopy images of hair after incubation of keratin-binding protein tagged with fluorescence dye and shampooing.

FIG. 10 shows a graph depicting thermal stability of keratin-binding protein at various temperatures.

FIG. 11 shows bioconjugation of lipid to keratin-binding protein via NHS-amine coupling.

FIG. 12 shows a MALDI-TOF profile and SDS-PAGE gel demonstrating the successful conjugation of lipid to keratin-binding protein (B22) by oxime ligation.

FIG. 13 shows MALDI profiles of KBPY, KBPY-lauric acid conjugate, and KBPY-oleic acid conjugate, confirming successful bioconjugation by NHS-coupling.

FIG. 14 shows a graph depicting the results of an IL-6 ELISA of KBP and KBP-lipid conjugate (top), and a graph depicting the results of an IFN ELISA of KBP and KBP-lipid conjugate (bottom). No protein was added to the cells as a negative control. Lipopolysaccharides (LPS) were used as a positive control.

FIG. 15 shows the results of a solubility test of KBP in oil-based solvents (polysorbate 40 (Tween® 40), Solubilisant CLR, glycerin, Solubilizer 611671, butylene glycol, polysorbate 20 (Tween® 20), hexylene glycol).

FIG. 16 shows a chart of water contact angles for four hair samples at varying levels of damage. The results indicate that virgin (undamaged) hair has a hydrophobic surface, due to covered cuticles and lipid.

FIG. 17 shows a graph comparing the degree of water contact angle for four hair samples which are treated with KBP-lipid conjugate. FIG. 17 also shows a chart of water contact angles for the four hair samples before and after treatment of KBP-lipid conjugates

FIG. 18 shows electron microscopy images of hairs before and after treatment with KBP and KBP-lipid conjugate and only treated with Tween® 40, all hairs were washed 2 times after treatment.

### DETAILED DESCRIPTION

The following description is merely intended to illustrate various embodiments of the invention. As such, specific embodiments discussed herein are not to be construed as limitations to the scope of the invention. It will be apparent to one skilled in the art that various changes or equivalents may be made without departing from the scope of the invention.

Hair is routinely damaged from ageing, environmental stress, heat styling, and use of chemical bleaches and dyes. The engineered keratin-binding constructs of the present disclosure form strong and long-lasting disulfide bonds and electrostatic bonds with keratin in hair, preventing and treating damaged hair. These strong and long-lasting bonds between the disclosed keratin-binding constructs and hair also make them ideal for use in hair dyes and compositions.

### Keratin Binding Molecules

Provided herein are engineered keratin-binding constructs that include at least one keratin-binding molecule. In some embodiments, the engineered keratin-binding construct includes one or more of each of two or more types of keratin-binding molecules.

The engineered keratin-binding construct can be represented by the formula., Xₙ-Yₘ, where X is one type of keratin-binding molecule, and Y is a different type of keratin-binding molecule, n=0−20 and m=0−20, wherein n and m cannot both be 0. Examples include X-Y, Y-X, X-X-Y, X-Y-X, X-Y-Y, X-X-Y-X, X-Y-X-Y, X-X-X-Y, Y-X-Y-X, Y-Y-X-X, X-X-X-Y-Y, etc.

The number and types of keratin-binding molecules is not limited, such that the engineered keratin-binding constructs can include 1, 2, 3, 4, 5, 6, 7, 8, 9, or more types of keratin-binding molecules.

Likewise, the number of any of the keratin-binding molecules is not limited, such that the number of any of the keratin-binding molecules can be 1, 3, 4, 5, 6, 7, 8, 9 or more.

Types of keratin binding molecule include keratin-binding proteins, antibody molecules that bind keratin, keratin-binding aptamers, or molecules selected by high throughput screening or directed evolution specifically for the property of binding to keratin.

Keratin-binding proteins may include, but are not limited to, trichoplein, pneumococcal serine rich repeat protein (PsrP), serine rich repeat protein (Srr-1), plectin, trichohyalin, myosin, TNF receptor 1-associated protein of TRADD, anti-Fas-binding factor 1 (Albatross), BMP, 14-3-3 proteins, plectin homology domain (TPHD), desmoplakin, the amino end of pinin, vitronectin, modified wheat gluten, pulmonary-associated surfactant protein D, p27kip1, primaquine and keratin binding polypeptides (see also Table 2).

Antibody molecules that bind keratin including monoclonal antibodies and antigen-binding fragments thereof, single domain antibodies such as VH, VL, VHH, and engineered constructs containing one or more of the antibodies, antigen-binding fragments thereof and/or single domain antibodies such as ScFv molecules, formatted camelid single variable domains, etc.).

The keratin-binding constructs disclosed herein may include one or more linker(s). The term “linker,” as used herein, refers to a chemical group or a molecule linking two molecules or moieties, e.g., two domains of a fusion protein, such as, for example, two keratin-binding domains. A linker may be, for example, an amino acid sequence, a peptide, or a polymer of any length and composition. Typically, the linker is positioned between, or flanked by, two groups, molecules, or other moieties and connected to each one via a covalent bond, thus connecting the two. In some embodiments, the linker is an amino acid or a plurality of amino acids (e.g., a peptide or protein). In some embodiments, the linker is an organic molecule, group, polymer, or chemical moiety. In some embodiments, the linker is 1-100 amino acids in length, for example, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. Longer or shorter linkers are also contemplated. For example, the linker may be a bond, one or more amino acids, a peptide, or a polymer, of any length and composition. In some embodiments, the linker is a Gly-Ser linker. In some embodiments, the linker comprises (GS)n (SEQ ID NO: 6), (GGS)n (SEQ ID NO: 7), (GGGS)n (SEQ ID NO: 8), (GGGGS)n (SEQ ID NO: 9), (G)n (SEQ ID NO: 10), (EAAAK)n (SEQ ID NO: 11), (SGGS)n (SEQ ID NO: 12), or (XP)n (SEQ ID NO: 13) motif, or a combination of any of these, wherein n is independently an integer between 1 and 30, and wherein X is any amino acid. It is to be understood that the linker lengths described as examples here are not meant to be limiting.

### Lipids

As disclosed herein, one or more lipid molecules can be conjugated to the one or more keratin binding moieties of the engineered keratin-binding constructs. Lipids that can be used in the engineered keratin-binding constructs include all lipid chains containing a linear chain of carbon with 6 to 20 carbons that is fully saturated, all lipid chains containing a linear chain of carbon of 6 to 20 carbons that contains one or more double bonds (degree of unsaturation), all lipid chains and derivatives thereof containing a sterol or its derivative, and all lipid chains and derivatives thereof containing cholesterol or its derivative.

Specific lipids that can be used in the engineered keratin-binding constructs include those in the following non-limiting list of specific lipid molecules: myristoleic acid/alcohol/amine, palmitoleic acid/alcohol/amine, sapienic acid/alcohol/amine, oleic acid/alcohol/amine, elaidic acid/alcohol/amine, vaccenic acid/alcohol/amine, linoleic acid/alcohol/amine, linoelaidic acid/alcohol/amine, alpha linolenic acid/alcohol/amine, arachidonic acid/alcohol/amine, eicosapentaenoic acid/alcohol/amine, erucic acid/alcohol/amine, caprylic acid/alcohol/amine (octanoic acid/alcohol/amine), lauric acid/alcohol/amine, myristic acid/alcohol/amine, palmitic acid/alcohol/amine, lignoceric acid/alcohol/amine, arachidic acid/alcohol/amine, stearic acid/alcohol/amine, and sphingolipids including ceramide, sphingosine, sphingomyelin, alpha cerebroside, gangliosides, sulfatides, and phytosphingosine.

In addition, derivatives of the aforementioned lipid compounds can be used in bioconjugation reactions for bioconjugation to the keratin binding molecules. For example, the lipid may contain an aminooxy group for bioconjugation to a protein, but other reactive groups can be used instead as will be known to those of skill in the art. In addition, the aforementioned lipid compounds can be combined into di or tri-functional molecules via conjugation to a small molecule linker (for example, triglycerides, glycerophospholipids, sphingolipids, and sterol lipids), and used in bioconjugation reactions for bioconjugation to the keratin binding molecules.

### Bioconjugation Reactions

Conjugates of keratin binding molecules to other molecules such as lipids or dyes, can be performed by a number of different reactions known in the art. For example, protein-lipid conjugates can be made by oxime formation, which produces a strong and stable bond between the protein and lipid. Oxime bioconjugation occurs in water and aqueous solvents and ambient environments. The protein molecule is stable during oxime chemistry bioconjugation. The resulting bioconjugated protein and lipid complex is stable on human hair through repeated hair washing.

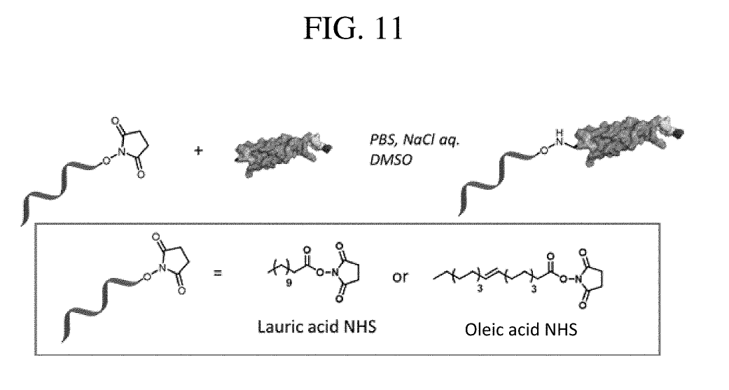

Other methods of bioconjugation can be used in the preparation of engineered keratin-binding constructs, for example, NHS-amine coupling of the lipid to KBP. FIG. 11, depicting the conjugation of an NHS-lipid and the N-terminal amine or alpha amine from a lysine residue, demonstrates additional functionalized lipids that can be used in conjugation of the lipids to engineered keratin-binding constructs. For example, lauric acid NHS or oleic acid NHS were conjugated to KBP as discussed below in Example 1. N-hydroxysuccinimide (NHS)-amine conjugation is a common and versatile technique for crosslinking proteins. The conjugation is highly reactive and results in a high yield. The conjugation can be carried in mild conditions and aqueous solutions, such as phosphate buffered saline (PBS).

Various additional reactions useful for bioconjugation with proteins are presented in Table 1 below. See also, Obermeyer and Olsen, ACS Macro Lett., 2015, 4(1): 101-110. In addition, small non-native/non-natural functional groups can be conjugated to the protein surface (e.g., enzymatically) for further functionalization. Some methodologies to modify these non-native/non-natural functional groups (bioorthogonal moieties), are shown in Scheme 2 of Obermeyer and Olsen, ACS Macro Lett., 2015, 4(1): 101-110.

TABLE 1

| Amino Acid | Bioconjugation Reaction Reagents |
|---|---|
| lysine | activated esters, reductive amination, |
| | thiazolidine-2-thione |
| glutamine, aspartic acid | amines and carbodiimides |
| cysteine | disulfides, maleimides, thiol-enes, |
| | dibromomaleimides and bis-sulfones |
| tyrosine | diazonium salts, Mannich-type coupling |
| | reagents, π-allylpalladium complexes, cyclic |
| | diazodicarboxylate reagents, |
| | phenylenediamine reagents |
| tryptophan | p-phenylenediamine reagents, rhodium |
| | carbenoids |
| N-terminus | oxidation/alkoxyamines, Pictet-Spengler |
| | reagents |

### Compositions

Compositions disclosed herein may include one or more of the keratin-binding constructs disclosed herein, in combination with one or more cosmetic ingredients, surfactants, preservatives, emulsifiers, softeners, moisturizers, humectants, hydrolyzed proteins, reconstructors, acidifiers, acidity regulators, detanglers, polymers, glossers, lubricants, sequestrants, antistatic agents, sunscreens, thermal protectors, conditioners, buffers, stabilizers, thickeners, salts, emollients, antioxidants, alcohols, polysorbates, PEGs, polyquaternium polymers, quarternary ammonium compounds, fragrances, dyes or colors, oils, esters, fatty acids, bioactive additives, food products, silicones, and water.

In some embodiments, the compositions disclosed herein may be daily or frequent use products including but not limited to shampoos, conditioners, gels, mousses, pomades, anti-frizz agents, sprays, or hair dyeing products that may be applied to the hair as part of customary hair care procedures including washing, conditioning, dyeing, drying, and styling.

In some embodiments, the compositions may be for use in a salon. In some embodiments, the compositions may be suitable for home use.

The compositions may be liquids, solids, or gels, and can be filled and stored in any suitable container, including bottles, cartons, tubes, and canisters. The compositions disclosed herein may also be provided or used as part of a kit. In some embodiments, the kit is a hair repair kit, hair treatment kit or hair coloring kit.

### Methods of Using the Engineered Keratin-Binding Constructs

The engineered keratin-binding constructs disclosed herein can be used to treat and/or repair damaged hair, prevent damage to hair, improve hair texture, moisture, shine and manageability. The above-described keratin-binding constructs may be used to treat, ameliorate, or improve hair that has suffered damage as a result of for example, sun damage, heat damage, chemical damage, and damage due to ageing. The engineered keratin-binding constructs may also be used prophylactically to prevent any such damage from occurring. The engineered keratin-binding constructs disclosed herein can be applied to the hair of a subject. The subject could be any mammal, preferably human. The engineered keratin-binding constructs disclosed herein can be applied by hand, applicator bottle, applicator brush, dropper, spray bottle, or by any other suitable method and/or applicator.

### Methods of Making the Engineered Keratin-Binding Constructs

The engineered keratin-binding constructs disclosed herein can be made by expression in cells, or by synthetic methods. For example, if the engineered keratin-binding construct comprises a polypeptide, the polypeptide can be produced in cells such as bacterial or yeast cells. This can be done by standard methods, such as cloning a sequence encoding the engineered keratin-binding construct into an expression plasmid, introducing the recombinant expression plasmid into a cell, expressing the engineered keratin-binding construct polypeptide in the cell, and isolating the engineered keratin-binding construct polypeptide from the cell.

Alternatively, the engineered keratin-binding construct polypeptide can be synthesized using chemical synthesis according to standard synthetic protocols.

Optionally one or more modification methods can be performed on the engineered keratin-binding construct polypeptide (made by any method), such as contacting the polypeptide with tyrosinase to convert tyrosine to melanin, contacting the polypeptide with appropriately modified (e.g., functionalized) lipid(s) in the presence of reagents that result in bioconjugation of the lipid(s) to the polypeptide, and/or contacting the polypeptide with appropriately modified (e.g., functionalized) dye molecule(s) in the presence of reagents that result in bioconjugation of the dye molecule(s) to the polypeptide.

For engineered keratin-binding constructs that comprise a polypeptide and a non-polypeptide molecule, such as when two keratin-binding molecules are combined and one is not a polypeptide, the polypeptide can be produced in cells such as bacterial or yeast cells, or produced by synthetic methods, and then joined to the non-polypeptide keratin-binding molecule using linkers appropriate for the respective chemistries of the polypeptide and non-polypeptide keratin-binding molecule. Similar modifications as described above for the keratin-binding construct polypeptide can then be made to the keratin-binding construct that comprises a polypeptide and a non-polypeptide molecule.

### EXAMPLES

### Example 1: Structure and Synthesis of Exemplary Engineered Keratin-Binding Construct

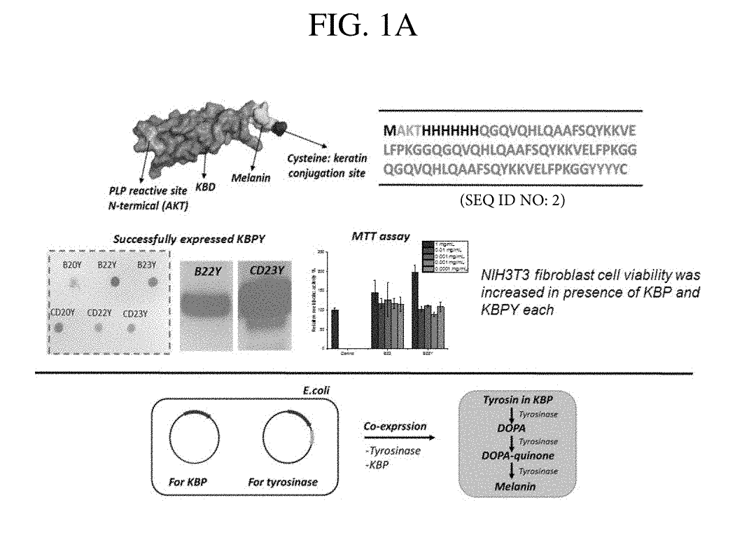

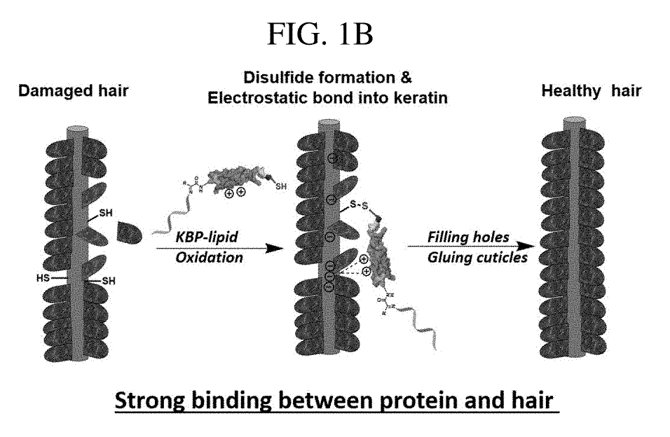

An exemplary engineered keratin-binding construct was produced having the features and sequence (MAKTHHHHHHQGQVQHLQAAFSQYKKVELFPKGGQGQVQHLQAAFSQYKKVELF PKGGQGQVQHLQAAFSQYKKVELFPKGGYYYYC; SEQ ID NO: 2) shown in FIG. 1A. A keratin-binding domain (QGQVQHLQAAFSQYKKVELFPKGG; SEQ ID NO: 1) was repeated three times and fused to an N-terminal methionine, a three amino acid pyridoxal 5-phosphate (PLP) reactive site (Ala-Lys-Thr, AKT), and a 6-His sequence (all before the N-terminus of the repeated keratin-binding domain), and a 4-Tyr sequence (YYYY; SEQ ID NO: 3) and a C-terminal Cys (all after the C-terminus of the repeated keratin-binding domain).

This engineered construct provides a smaller molecule that can penetrate deep into hair fibers. The construct provides electrostatic interaction via the keratin-binding domain (hair carries a negative charge) and disulfide bonding to keratin proteins in hair via the C-terminal cysteine (see FIG. 1B).

In addition, the N-terminal amine is a site selective site for chemistry, providing a site for conjugation of lipid molecules (FIG. 1B). The tyrosine residues can be converted to melanin using tyrosinase (FIG. 1A), for color dyeing.

A nucleic acid molecule encoding the exemplary engineered keratin-binding construct was prepared and cloned into a plasmid (“For KBP” in FIG. 1A), which was introduced into E. coli cells. FIG. 1A also shows optional expression of tyrosinase encoded on another plasmid (“For tyrosinase” in FIG. 1A) in the E. coli cells. Co-expression of the exemplary engineered keratin-binding construct and tyrosinase results in conversion of tyrosine residues in the exemplary engineered keratin-binding construct (“KBP”) to melanin according to the scheme shown on the lower right side of FIG. 1A. Alternatives for this synthesis include chemical synthesis of the exemplary engineered keratin-binding construct by standard peptide synthesis, with or without tyrosinase treatment to convert tyrosine to melanin; and treating the exemplary engineered keratin-binding construct produced in cells with tyrosinase after purification of the exemplary engineered keratin-binding construct from the cells.

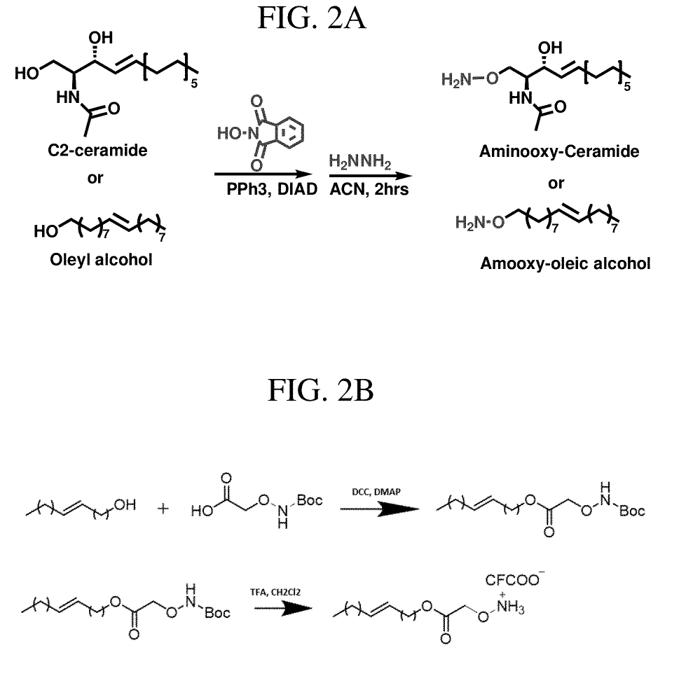

FIGS. 2A and 2B show schemes for preparation of functionalized lipids that can be used in conjugation of the lipids to engineered keratin-binding construct. For example, the functionalized lipids are attached to the PLP reactive site of the exemplary engineered keratin-binding construct. Upon treatment of hair with the engineered keratin-binding construct, the conjugated lipids provide a hydrophobic protective layer for the hair surface. Ceramide can be used for gluing cuticles on the hair surface.

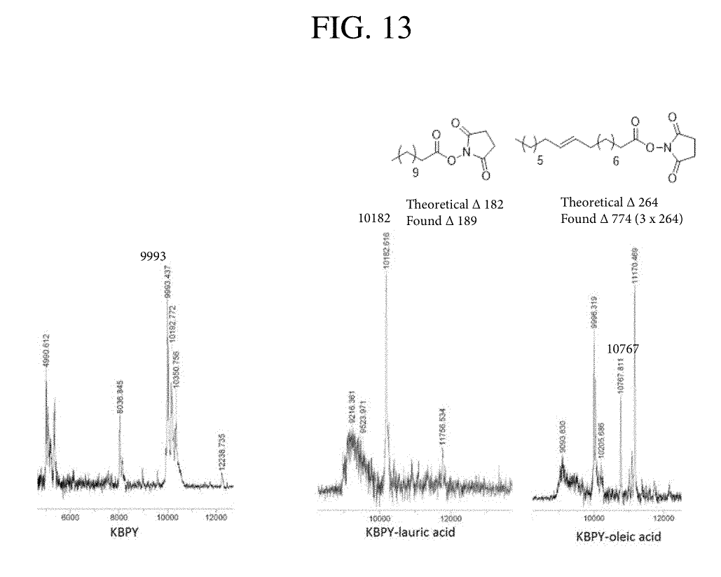

FIG. 11 depicts bioconjugation of lipids with B22Y (KBPY) using NHS-amine coupling. Lauric acid and oleic acid were separately conjugated to KPB using NHS. NHS-lipids were diluted in DMSO, and the reactions were carried out in aqueous solution of NaCl and PBS. NHS coupling of the functionalized lipids to the N-terminal amine or lysine of KBPY were confirmed by MALDI analysis, as shown in FIG. 13.

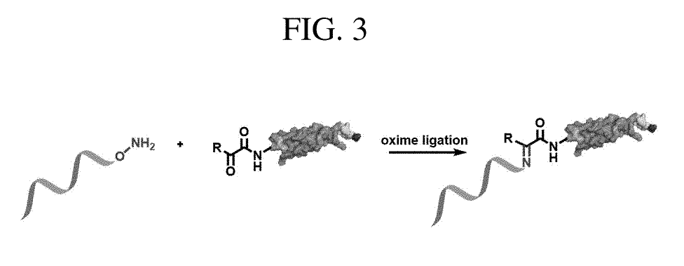

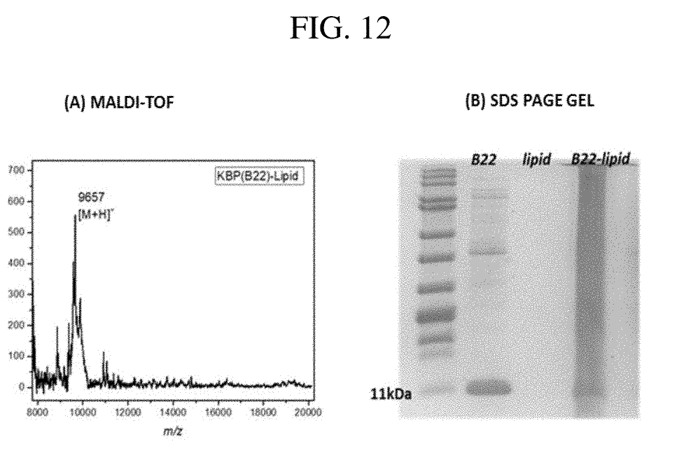

FIG. 3 shows oxime bioconjugation between a functionalized lipid and the PLP reactive site of the exemplary engineered keratin-binding construct. Oxime bioconjugation occurs in water solvent and ambient environment, and produces a strong and stable bond. The exemplary engineered keratin-binding construct is stable during the oxime chemistry. The bioconjugated protein—lipid complex is stable on human hair through repeated hair washing. Conjugation of keratin-binding protein (B22) to the lipid was also confirmed by MALDI and SDS-PAGE gel, as shown in FIG. 12.

### Example 2: Treatment of Damaged Hair

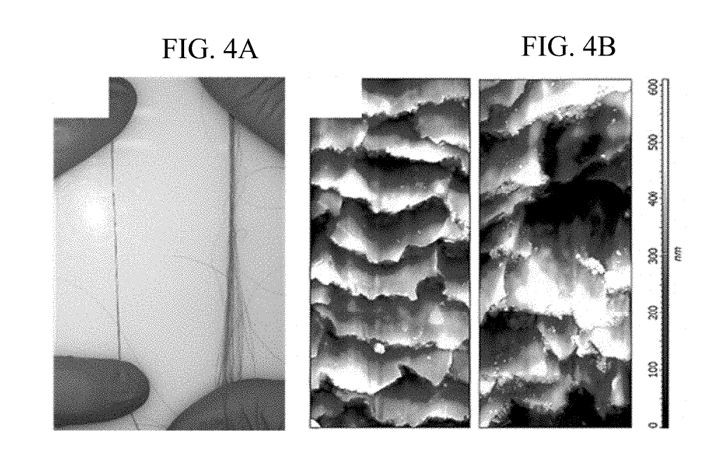

Chemically damaged hair shows bleached color and cuticle loss. As shown in FIGS. 4A and 4B, damaged hair has loss of cuticles and a bleached color,

To provide damaged hair for treatment using the exemplary engineered keratin-binding construct, hair was incubated with 10% H2O2(v/v) in 0.1M Na2CO3/NaHCO3 (pH 9.0) buffer at 50° C. for 1 hr, and then the hairs were washed in deionized (DI) water.

For visualization of the effects of the exemplary engineered keratin-binding construct and demonstration of hair absorption, keratin-binding domain (“peptide1” and “peptide2”) were conjugated to FITC as shown below for peptide2:

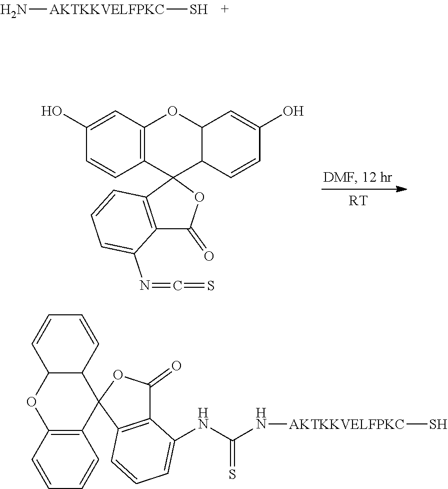

Peptide1 is a keratin binding domain (AKTKKVELFPK; SEQ ID NO: 4). Peptide2 has the same keratin binding domain as peptide 1 plus a C-terminal cysteine (AKTKKVELFPKC; SEQ ID NO: 5).

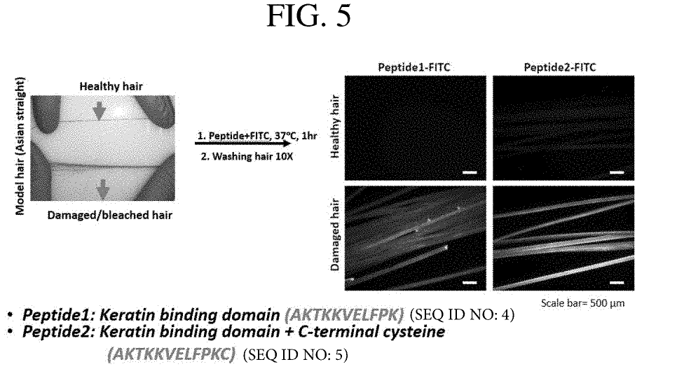

Samples of healthy and damaged hair were incubated with the FITC-conjugated peptides at 37° C. for 1 hr, followed by washing the hair 10 times (FIG. 5). The results are provided in the images obtained by fluorescent microscopy shown in the right part of FIG. 5.

Damaged hair absorbed more of the FITC-labeled keratin binding peptides compared to healthy hair. Overall, peptide2-FITC bound to hair more than peptide1-FITC due to both physical and chemical interactions. Neither peptide1-FITC nor peptide1-FITC is shown as binding to healthy hair due to hydrophobic surfaces (hair cuticles). Compared to healthy undamaged hair, more labeled peptide was absorbed by damaged hair as shown in the fluorescence microscope images. This is because damaged hair has exposed keratin proteins.

### Example 3: Expression of Exemplary Engineered Keratin-Binding Construct

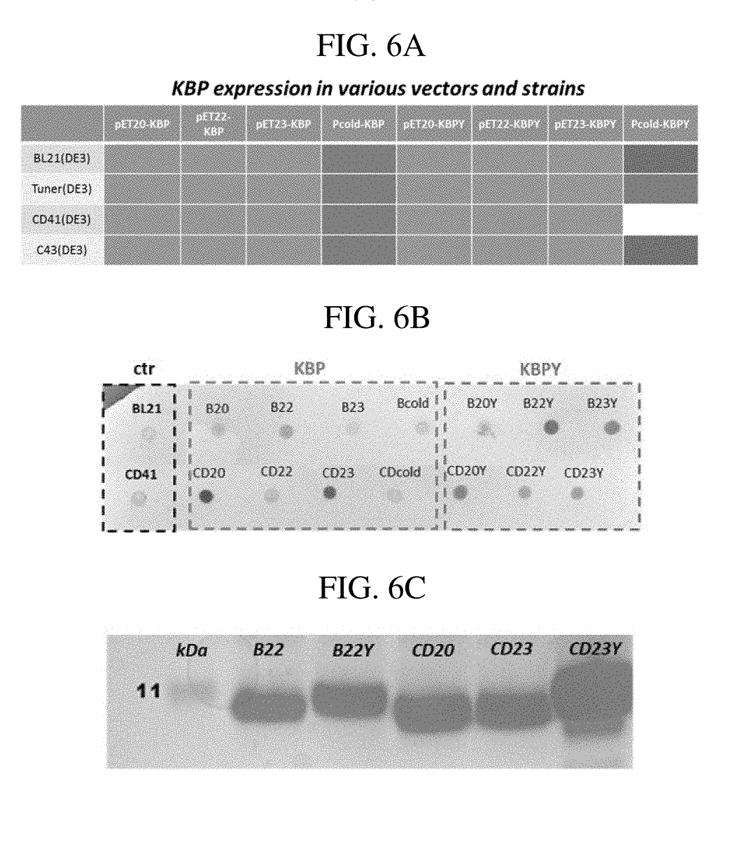

Several different plasmids and cell types were assessed for optimal expression of the exemplary engineered keratin-binding construct. The sequence encoding the keratin-binding protein, with (“KBPY”) or without (“KBP”) tyrosine residues, was cloned into the plasmids pET20, pET22, pET23, and pCold. See FIGS. 6A-6C. These plasmids were introduced into the following E. coli strains BL21(DE3), Tuner(DE3), CD41(DE3) and C43(DE3). The matrix of plasmids, cells and keratin-binding proteins is shows in FIG. 6A. FIGS. 6B and 6C are images of dot blots and an SDS-PAGE gel, respectively, showing expression of the keratin-binding proteins with high yield by different combinations of cell lines and plasmids.

### Example 4: Bioconjugation of Dye to the Exemplary Engineered Keratin-Binding Construct

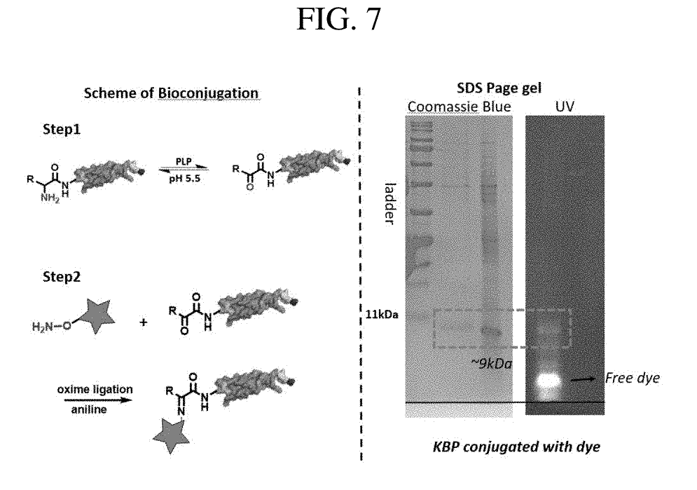

Dye molecule (represented by a star) was conjugated to the exemplary engineered keratin-binding construct in a two-step process shown in FIG. 7 (left panel). The resulting conjugate was run on an SDS-PAGE gel, which was stained with Coomassie Blue. The right panel of FIG. 7 shows the Coomassie Blue-stained gel, and a UV image of the same gel showing the presence of conjugated dye (dotted line box) and free dye.

### Example 5: Affinity of Keratin-Binding Protein for Damaged Hair

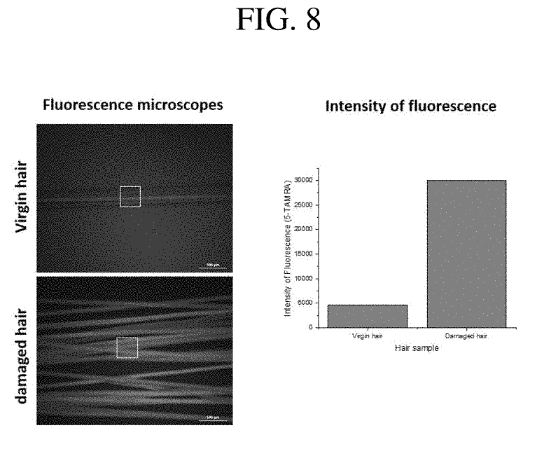

Keratin binding protein tagged with a fluorescent dye was incubated with damaged and undamaged (virgin) hair, and observed by fluorescence microscopy. The results are depicted in FIG. 8, with microscopy images shown on the left and intensity of corrected fluorescence shown on the right. Corrected fluorescence=Integrated Density−(selected area X Mean fluorescence of background).

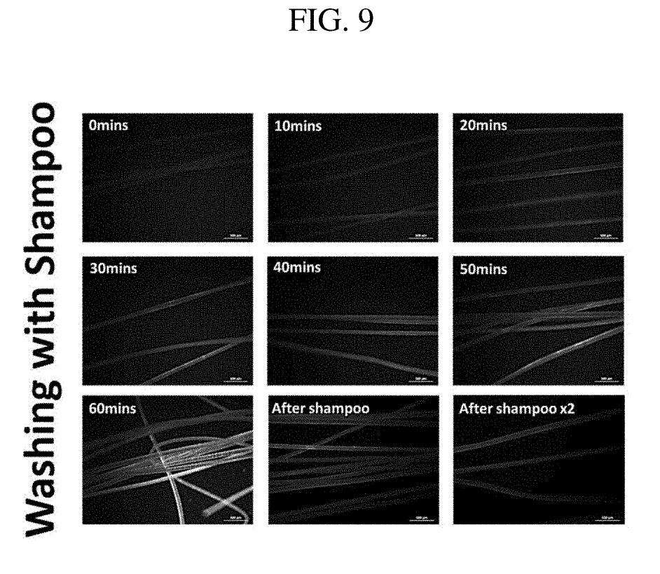

The damaged hair incubated with keratin binding protein tagged with a fluorescent dye for varying amounts of time was then subjected to washing with shampoo. The results of fluorescence microscopy are shown in FIG. 9. KBP binds more readily to damaged hair than healthy hair due to the exposed cysteines on damaged hair.

### Example 6: Thermal Stability of Keratin-Binding Protein

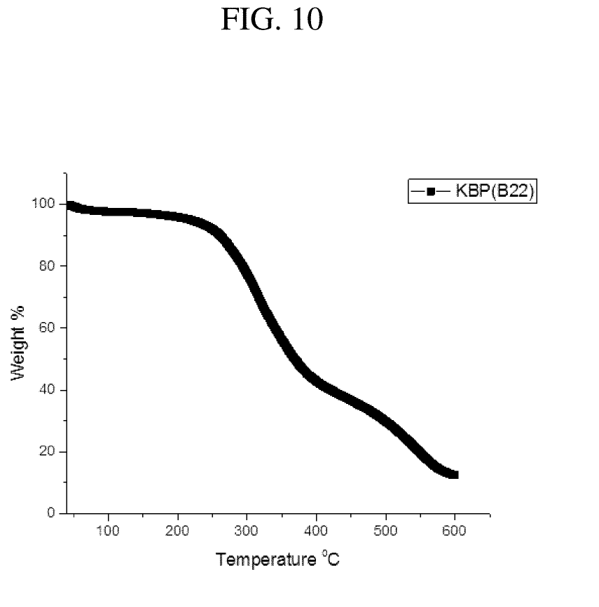

Keratin-binding protein, with (“B22Y”) or without (“B22”) tyrosine residues, was tested for thermal stability. Weight was measured as a function of increasing temperature. The results depicted in FIG. 10 show that keratin-binding protein is stable above 100° C.

### Example 7: Immunogenicity of Keratin-Binding Protein (KBP) and KBP-Lipid Conjugates

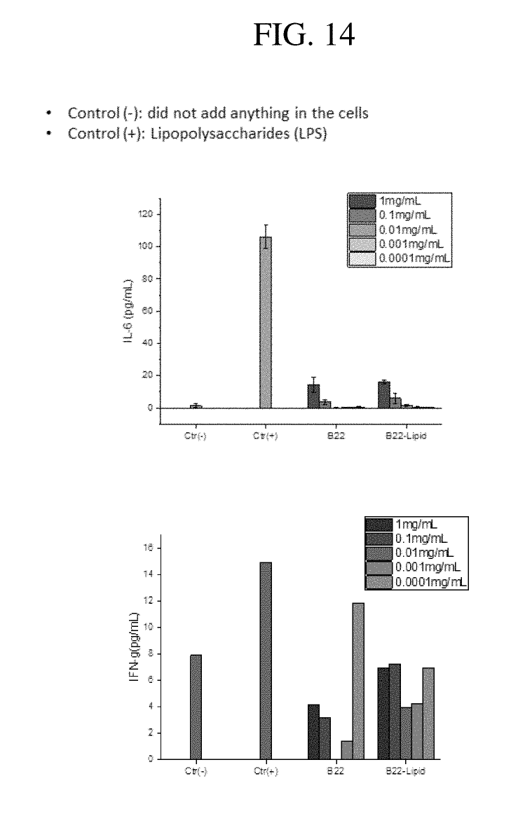

Keratin-binding protein (B22) was tested for immune response by performing an IL-6 ELISA and IFN-γ ELISA. The effects of KBP and KBP-lipid conjugate on the immune response was assessed by measuring levels of IL-6 and IFN-γ in fibroblast cells. No protein was added to the cells as a negative control. Lipopolysaccharides (LPS) were used as a positive control. No production of IL-6 or IFN-γ was recorded, as shown in FIG. 14.

### Example 8: Solubility of Keratin-Binding Protein

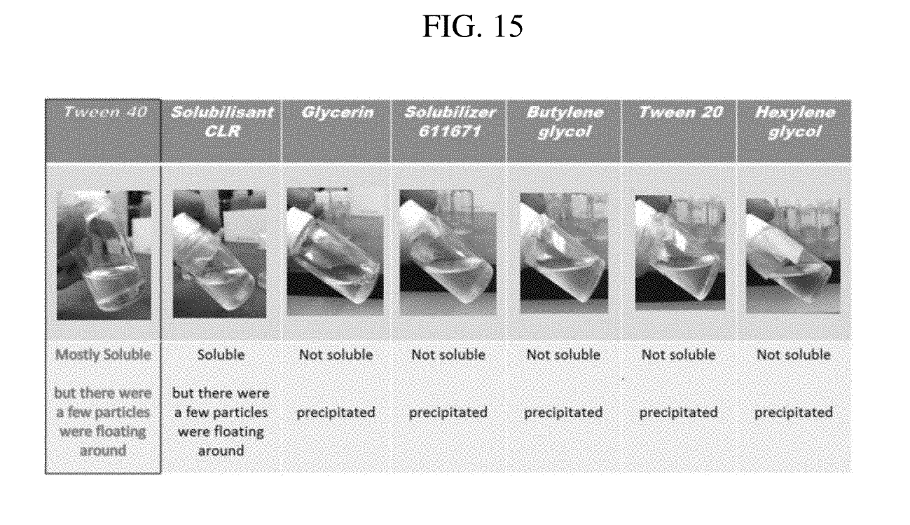

Solubility tests were performed by adding keratin-binding protein to 7 different solvents at concentrations of 2.5 mg protein/10 mL solvent. As shown in FIG. 15, KBP was not soluble in glycerin, butylene glycol, hexylene glycol, polysorbate 20 (Tween® 20), or Solubilizer 611671. KBP was soluble in polysorbate 40 (Tween® 40) and Solubilisant CLR, with only a few visible particles present in solution.

### Example 9: Effects of Keratin-Binding Protein on Hair

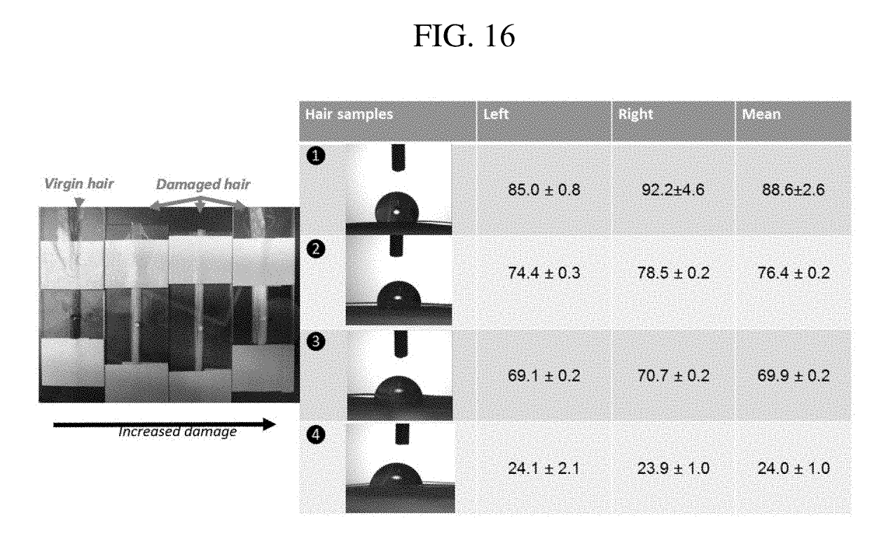

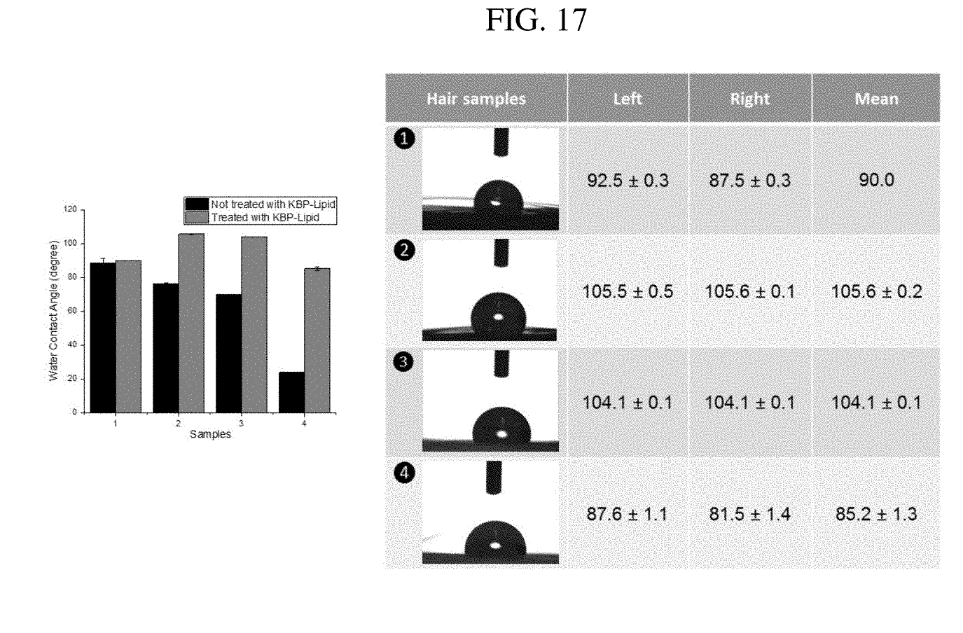

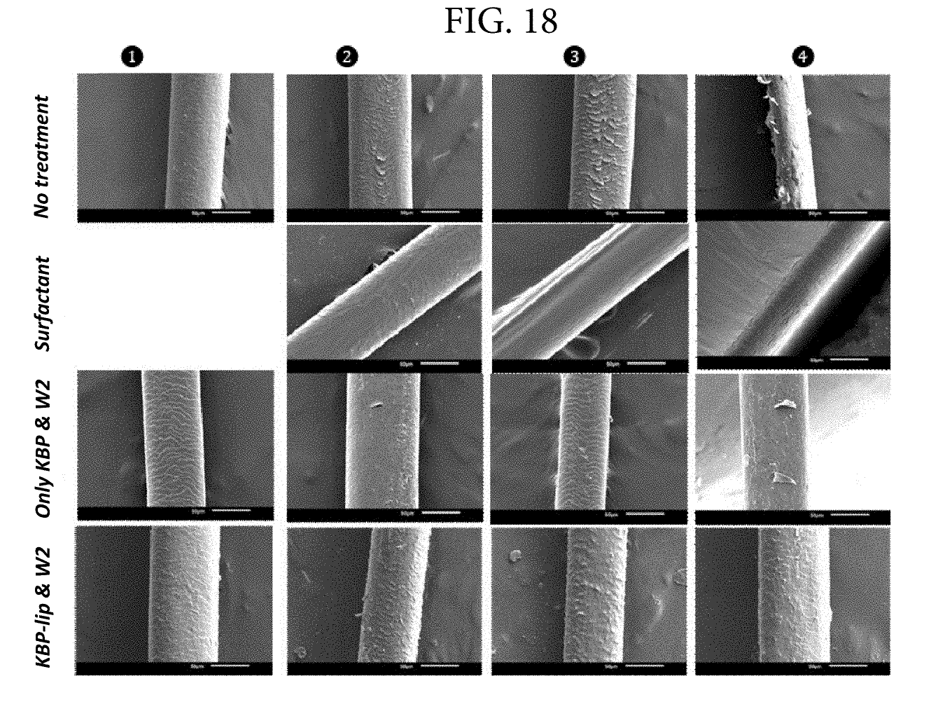

Undamaged (“virgin”) hair has intact, covered cuticles, and thus a more hydrophobic surface. The effects of keratin-binding protein on the hair was assessed by measuring the water contact angle, as the water contact angle varies depending on the level of damage to the hair follicle. The water contact angles for damaged and undamaged hair tested are shown in FIGS. 16 ad 17.

Samples of untreated hair, hair treated with surfactant, hair treated with keratin-binding protein, and hair treated with a KBP-lipid conjugate were examined using scanning-electron microscopy. The results are depicted in FIG. 18.

TABLE 2
Keratin-binding molecules and reference therefor

| | Reference |
|---|---|
| Protein | |
| trichoplein | J Cell Sci. 2005 Mar. 1; 118(Pt 5): 1081-90. |
| pneumococcal serine rich repeat | Open Biol. 2014 Jan. 15; 4: 130090. doi: |
| protein (PsrP) | 10.1098/rsob.130090. |
| serine rich repeat protein-1 (Srr-1) | https://doi.org/10.1016/j.ijbiomac.2014.12.048 |
| plectin | http://jcb.rupress.org/content/jcb/183/1/19.full.pdf |
| trichohyalin | http://jcs.biologists.org/content/118/5/1081 |
| myosin | Journal of Cell Science 118, 1081-1090 |
| TNF receptor 1-associated protein | http://jcs.biologists.org/content/joces/118/5/1081.full.pdf |
| of TRADD | |
| Anti-Fas-binding factor 1 | J Cell Biol. 2008 Oct. 6; 183(1): 19-28 |
| (Albatross) | |
| BMP | http://journals.plos.org/plosone/article?id=10.1371/journal.pone.0137233 |
| 14-3-3 proteins | https://www.ncbi.nlm.nih.gov/pmc/articles/PMC123655/pdf/pq0702004373.pdf |
| plectin homology domain (TPHD) | https://www.ncbi.nlm.nih.gov/pmc/articles/PMC2557036/pdf/jcb1830019.pdf |
| Desmoplakin | J Cell Biol. 1993 November; 123(3): 691-705. |
| The amino end of pinin | http://www.jbc.org/content/275/20/14910.full |
| Vitronectin | v1tronectin binds to uratin filaments |
| modified wheat gluten | DOI: 10.1098/rsos.171216 |
| pulmonary-associated surfactant | https://onlinelibrary.wiley.com/doi/epdf/10.1002/prot.24037 |
| protein D | |
| p27kip1 | https://www.aaas.org/abstract/keratin-17-maintains- |
| | proliferation-binding-p27kip1-and-facilitating-its- |
| | nuclear-export |
| Small molecules | |
| Primaquine | http://europepmc.org/abstract/med/12711178 |
| keratin binding polypeptides | https://patentscope.wipo.int/search/en/detail.jsf?docId= |
| | WO2005115306&recNum=1&maxRec=&office=& |
| | prevFilter=&sortOption=&queryString=&tab=PCT+ |
| | Biblio; https://patents.justia.com/patent/20090156485 |

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. An engineered keratin-binding construct comprising at least one keratin-binding molecule, optionally comprising one or more of each of two or more types of keratin-binding molecule.

2. (canceled)

3. The engineered keratin-binding construct of claim 1, wherein the engineered keratin-binding construct comprises the formula Xₙ-Yₘ, where X is one type of keratin-binding molecule, and Y is a different type of keratin-binding molecule, n=0−20 and m=0−20, wherein n and m cannot both be 0.

4. The engineered keratin-binding construct of claim 1, wherein at least one type of the at least one keratin-binding molecule is a keratin-binding protein, optionally wherein all of the types of the at least one keratin-binding molecule are keratin-binding proteins, optionally wherein the keratin-binding protein comprises 1-5 repeats of an amino acid sequence (QGQVQHLQAAFSQYKKVELFPKGG) set forth in SEQ ID NO: 1.

5.-6. (canceled)

7. The engineered keratin-binding construct of claim 1, further comprising a site for bioconjugation
optionally wherein the site for bioconjugation is for bioconjugation of a lipid,
optionally wherein the site for bioconjugation is located at or near an end of the engineered keratin-binding construct,
optionally wherein at least one keratin-binding molecule is a protein, and the site for bioconjugation is located at the N-terminus of the protein or within 5 amino acid residues from the N-terminus of the protein,
optionally wherein the site for bioconjugation comprises a pyridoxal 5-phosphate (PLP) reactive site, optionally wherein the PLP reactive site comprises the amino acid sequence alanine-lysine-threonine (AKT).

8.-12. (canceled)

13. The engineered keratin-binding construct of claim 1, further comprising a keratin conjugation site
optionally wherein the keratin conjugation site comprises one or more cysteines,
optionally wherein the keratin conjugation site is located at or near an end of the engineered keratin-binding construct,
optionally wherein at least one keratin-binding molecule is a protein, and the keratin conjugation site is located at the C-terminus of the protein,
optionally wherein at least one keratin-binding molecule is a protein, and wherein the one or more cysteines is part of a single polypeptide chain with the keratin-binding molecule.

14.-17. (canceled)

18. The engineered keratin-binding construct of claim 1, further comprising a melanin precursor
optionally wherein the melanin precursor comprises one or more tyrosines,
optionally wherein the one or more tyrosines comprise 4-20 tyrosines,
optionally wherein at least one keratin-binding molecule is a protein, and wherein the one or more tyrosines is part of a single polypeptide chain with the keratin-binding molecule.

19.-21. (canceled)

22. The engineered keratin-binding construct of claim 1, further comprising melanin or a hair dye.

23. The engineered keratin-binding construct of claim 1, further comprising a lipid
optionally wherein the lipid is conjugated to the site for bioconjugation, optionally wherein the site for bioconjugation is the amino acid sequence AKT, and wherein the lipid is conjugated to the lysine of the amino acid sequence AKT,
optionally wherein the lipid is selected from the group consisting of myristoleic acid/alcohol/amine, palmitoleic acid/alcohol/amine, sapienic acid/alcohol/amine, oleic acid/alcohol/amine, elaidic acid/alcohol/amine, vaccenic acid/alcohol/amine, linoleic acid/alcohol/amine, linoelaidic acid/alcohol/amine, alpha linolenic acid/alcohol/amine, arachidonic acid/alcohol/amine, eicosapentaenoic acid/alcohol/amine, erucic acid/alcohol/amine, caprylic acid/alcohol/amine (octanoic acid/alcohol/amine), lauric acid/alcohol/amine, myristic acid/alcohol/amine, palmitic acid/alcohol/amine, lignoceric acid/alcohol/amine, arachidic acid/alcohol/amine, stearic acid/alcohol/amine, and sphingolipids including ceramide, sphingosine, sphingomyelin, alpha cerebroside, gangliosides, sulfatides, and phytosphingosine.

24.-26. (canceled)

27. A composition comprising the engineered keratin-binding construct of claim 1.

28.-29. (canceled)

30. A nucleic acid molecule encoding the engineered keratin-binding construct of claim 1.

31. A vector comprising the nucleic acid molecule of claim 30.

32. A cell comprising the nucleic acid molecule of claim 30.

33. The cell of claim 32, wherein the cell is a bacterial cell or a yeast cell, optionally wherein the bacterial cell is an E. coli cell.

34. (canceled)

35. A method of improving or repairing damage to hair comprising
applying the engineered keratin-binding construct of claim 1 to the hair for a time sufficient to improve or repair the damage to the hair.

36.-39. (canceled)

40. A method of coloring or dyeing hair comprising
applying the engineered keratin-binding construct of claim 1 to the hair for a time sufficient to color or dye the hair, wherein the engineered keratin-binding comprises melanin or a hair dye molecule conjugated to the at least one keratin-binding molecule.

41.-44. (canceled)

45. A method for producing an engineered keratin-binding construct comprising
expressing the engineered keratin-binding construct of claim 1 in a cell, or
chemically synthesizing the engineered keratin-binding construct of claim 1.

46.-47. (canceled)

48. A method for producing an engineered keratin-binding construct comprising a lipid, comprising
expressing the engineered keratin-binding construct of claim 1 in a cell or chemically synthesizing the engineered keratin-binding construct of claim 1, and,
conjugating a lipid to the engineered keratin-binding construct.

49.-50. (canceled)

51. A method for preparing a hair dye composition comprising
expressing or chemically synthesizing the engineered keratin-binding construct of claim 1, wherein the engineered keratin-binding construct comprises a melanin precursor; and
contacting the engineered keratin-binding construct with tyrosinase to convert the melanin precursor to melanin.

52.-56. (canceled)

57. A method for preparing a hair product composition comprising combining the engineered keratin-binding construct of claim 1 with one or more cosmetic ingredients.

58. (canceled)
